(19) United States
(12) Patent Application Publication
(10) Pub. No.: US 2001/0021390 A1
(43) Pub. Date: Sep. 13, 2001

(54) Prolongation of chemical activity of flavorants and therapeutics on mucin coated surfaces using mucin adhering amphiphilic substances

(75) Inventor: John R. Lau, Howard, OH (US)

Correspondence Address: William Schmonsees, Heller Ehrman White & McAuliffe LLP, 275 Middlefield Road, Menlo Park, CA, 94025-3506, US

(73) Assignee: SDG, Inc.

(21) Appl. No.: 09/774,484

(22) Filed: Jan. 30, 2001

Related U.S. Application Data
(63) Continuation of application No. 08/315,359, filed on Sep. 30, 1994, now Pat. No. 6,207,192.

(51) Int. Cl. A61K 9/70; A61F 13/00

(52) U.S. Cl. 424/443

(57) ABSTRACT

Cetyl pyridinium chloride and other amphiphilic substances may be mixed with flavorants or therapeutics in accompaniment with bulking agents or sweeteners in order to prolong their duration of action at the site of attachment, which is a mucin coated surface for the purpose of providing the consumer with a more efficacious product.

### BACKGROUND OF THE INVENTION

Historically, it has been a goal of the flavorant industry and of pharmaceutical companies to maintain the action of flavorants and therapeutics upon administration to mucin coated surfaces of a human host. Recent marketing strategies of these companies have attempted to focus on providing the consumer with a safe and effective product that is convenient to use and has a prolongation of action. However, in the absence of achieving a superior product with a distinct advantage, these strategies have been met with consumers who are dissatisfied with performance. For example, previous to the findings of this invention, the consumer could only perceive the flavor of a breath mint or the performance of a throat lozenge lasting for a duration of twenty minutes. It is a consensus of those in the flavorant industry that a breath mint product with a prolonged duration of action, of up to an hour or longer, would provide the consumer an increased perception of flavor satisfaction and present a true marketing advantage. Likewise, with pharmaceutical products, a more efficacious pharmaceutical would increase the therapeutic index by prolonging pharmaceutical action. This effect would be beneficial especially during periods of sleep or during periods of intense activity when product administration becomes cumbersome. This invention addresses and solves some of these problems with products that have a pronounced prolongation of action and adhere to mucin coated surfaces.

### SUMMARY

This invention utilizes amphiphilic substances to adhere to mucin surfaces and provides a hydrophobic reservoir which can entrap and sequester organic compounds, comestibles, and therapeutic agents in order to prolong their release and duration of action in a warm-blooded host.

### OBJECTS OF THE INVENTION

It is an object of the invention to utilize an additive substance in a breath mint formulation to prolong the retention of flavorant taste in the oral cavity of a warm-blooded host.

Another object of the invention is to incorporate the additive substance in a throat lozenge to prolong the action of an anesthetic or therapeutic substance in the oral cavity and esophagus of a warm-blooded host.

Another object of the invention is to employ an additive substance to maintain the adherence of the product to the oral cavity, esophagus, eye, vagina, bladder, rectal cavity, stomach, and mucin coated surfaces in a warm-blooded host.

Another object of the invention is to utilize an additive substance for a time-released application of the product.

It is still a further object of the invention to utilize an additive substance for the sequestering of organic compounds and to prolong the duration of action of said organic compounds in a warm-blooded host.

### DRAWINGS

FIG. 1 is a representation of the structural formula of the amphiphilic molecule, cetyl pyridinium chloride. The symbol for the hydrophilic head group is represented by (≡) and the symbol for the hydrophobic tail group is represented by (-).

FIG. 2a is a diagrammatic representation of a spherical micelle cross-section.

FIG. 2b is also representative of a spherical micelle cross-section. The individual amphiphilic molecular constituents are depicted using the structural formula of CPC.

FIG. 3 illustrates a cross-section of a liposome depicting the bipolar lipid membrane structure with individual amphiphilic molecular constituents.

FIG. 4a depicts individual molecule of CPC, using structural formulas, which are individually bound to a mucin coat. The hydrophobic hydrocarbon tails of CPC are oriented toward the bulk aqueous phase and are positioned distally to the mucin coat. The molecules of CPC are arranged in a collective manner in a linear array.

FIG. 4b illustrates the same phenomena as FIG. 4a except that ρ-aminobenzoic acid ethyl aster, represented by the symbol (▴), is interspersed and trapped between the ordered hydrophobic chains of CPC.

FIG. 5 shows a graph which depicts the plot of the variation in physical properties of an amphiphile as a micelle concentration is the point at which the structure of the amphiphile changes from the unassociated or free state to the micellar form.

FIG. 6 is a representation of the structural formula for the topical anesthetic ρ-aminobenzoic acid ethyl aster.

FIG. 7 shows a graph in which the percent of ρ-aminobenzoic acid ethyl aster, adhering to a mucin agarose gel, is plotted as a function of the number of 1.0 ml, 10 mM phosphate buffer pH 7.0 washes performed at ambient temperature. The legend (▴-▴) represents the control data where only ρ-aminobenzoic acid ethyl aster is added to the gel. The legend (▪-▪) represents the experimental data where ρ-aminobenzoic acid ethyl ester in the presence of CPC is added to the gel.

### PREFERRED EMBODIMENT

This disclosure describes the unexpected discovery that molecules of cetyl pyridinium chloride (CPC) can adhere to mucin coated surfaces and function collectively to sequester organic molecules and comestibles, such as flavorants, and also pharmaceuticals or therapeutics. These organic molecules can be slowly released over time, thereby, prolonging their duration of action. It has been demonstrated experimentally that flavorants such as menthol, and therapeutics such as the topical anesthetic ρ-aminobenzoic acid ethyl ester, can be sequestered within the hydrophobic regions of a CPC matrix. When the combination of CPC and the intended organic is administered to a warm-blooded host, strong adherence to mucin and a prolongation of action of the organic is achieved. Examples of classes of therapeutics that may be used in this invention are as follows: anesthetics, analgesics, antibiotics, antifungals, antitoxins, antihistamines, antineoplastics, anthelminthics, bronchial dilators, and ophthalmics.

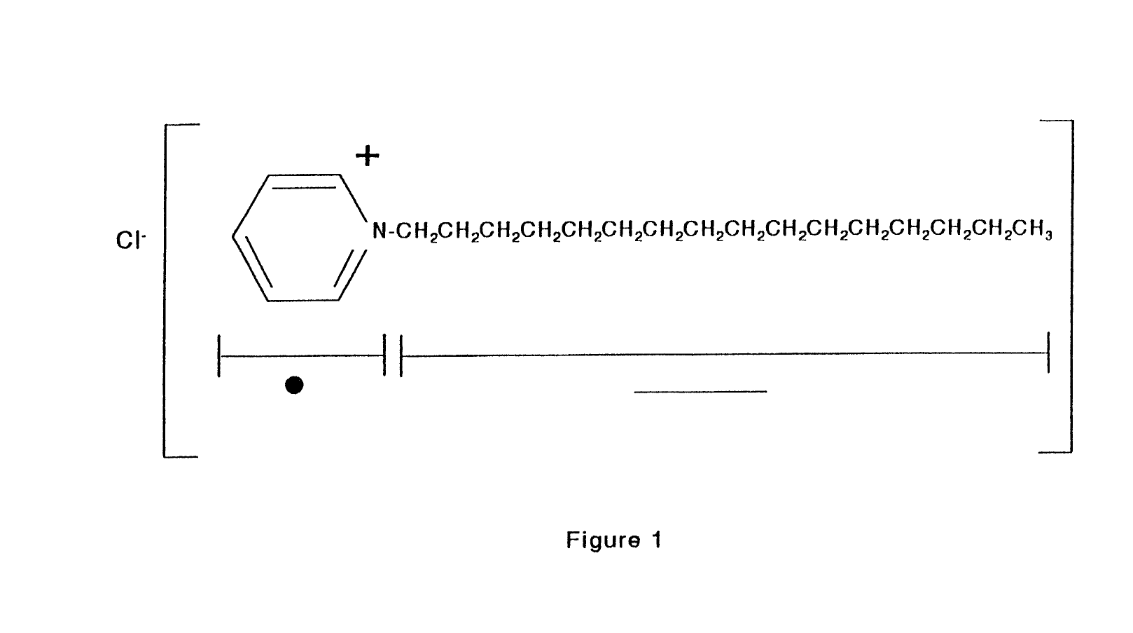

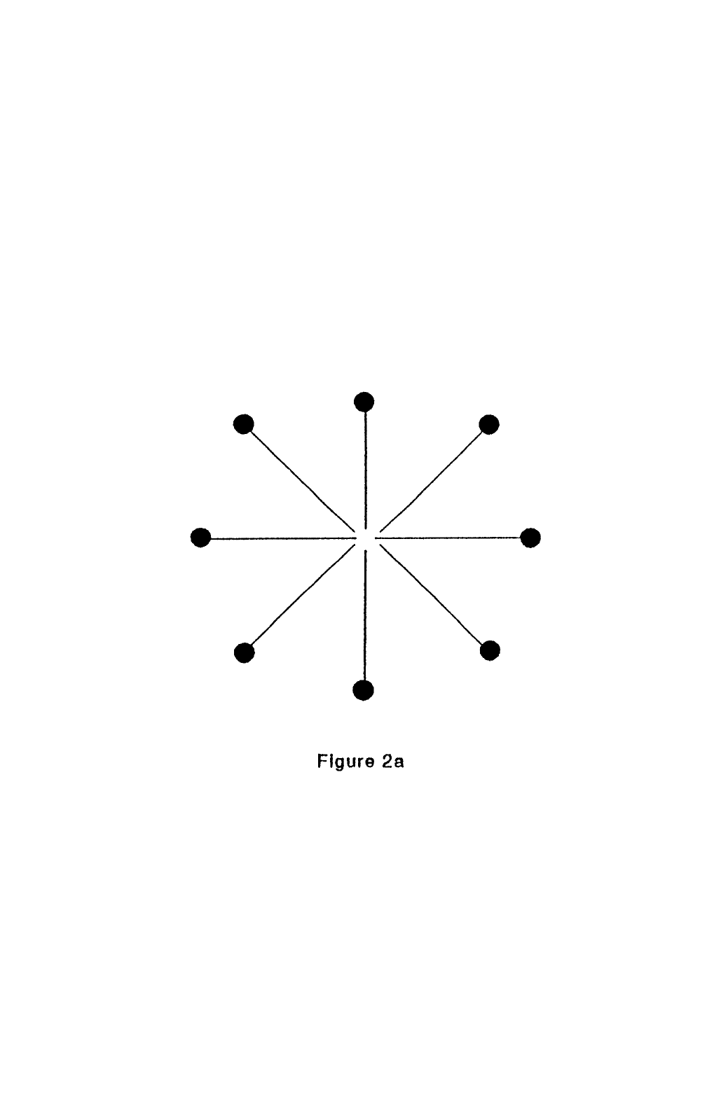

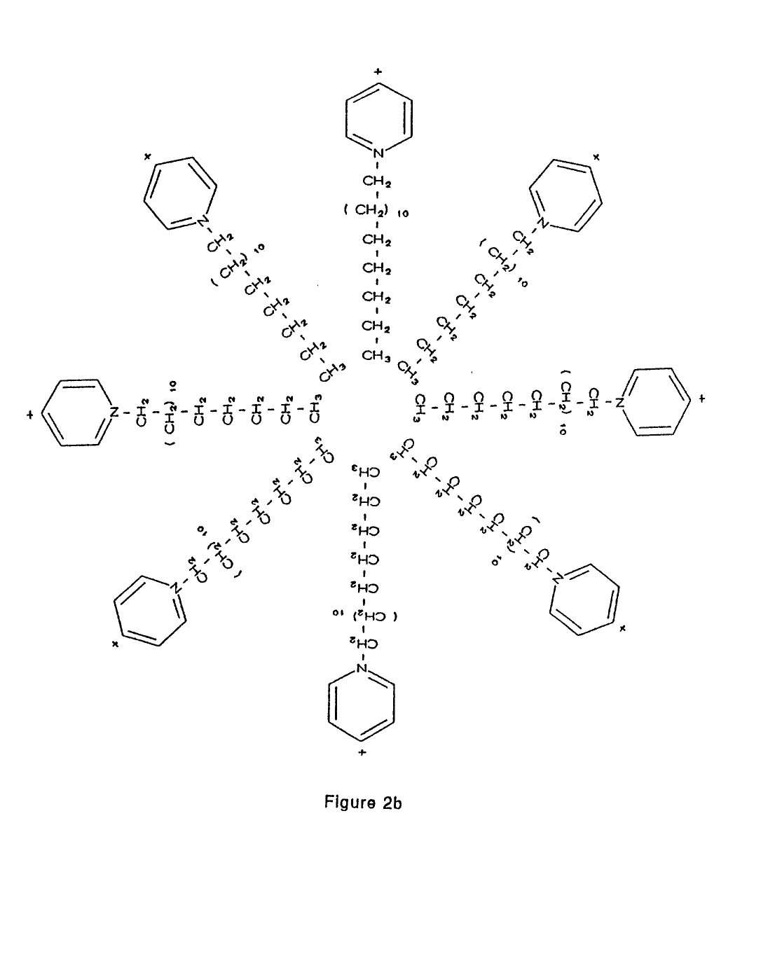

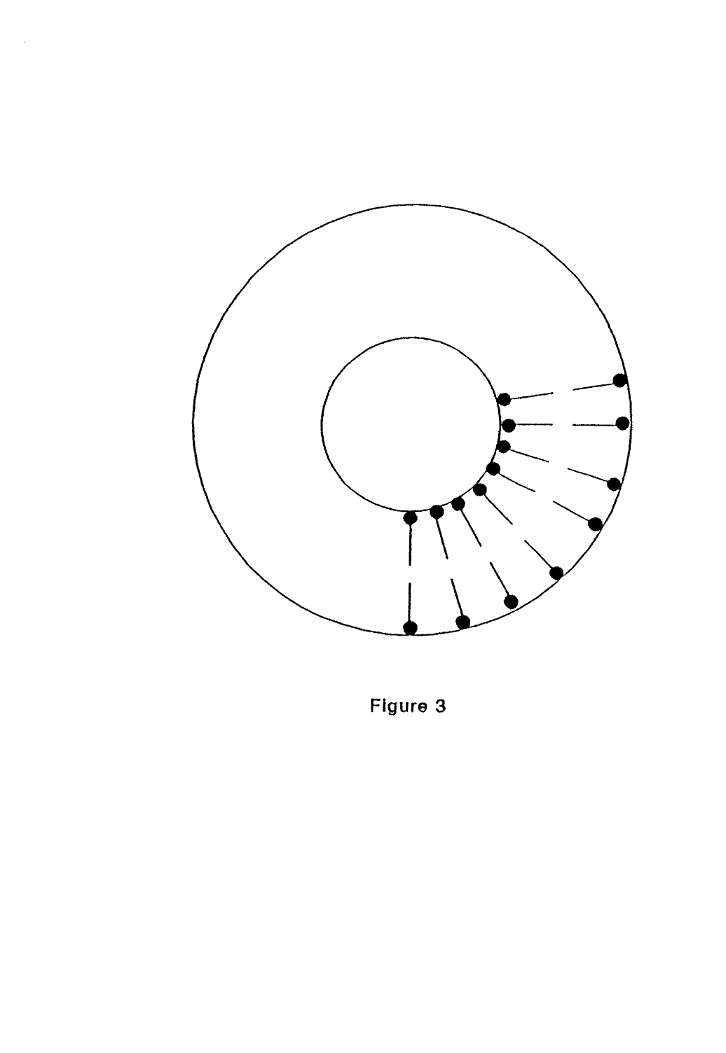

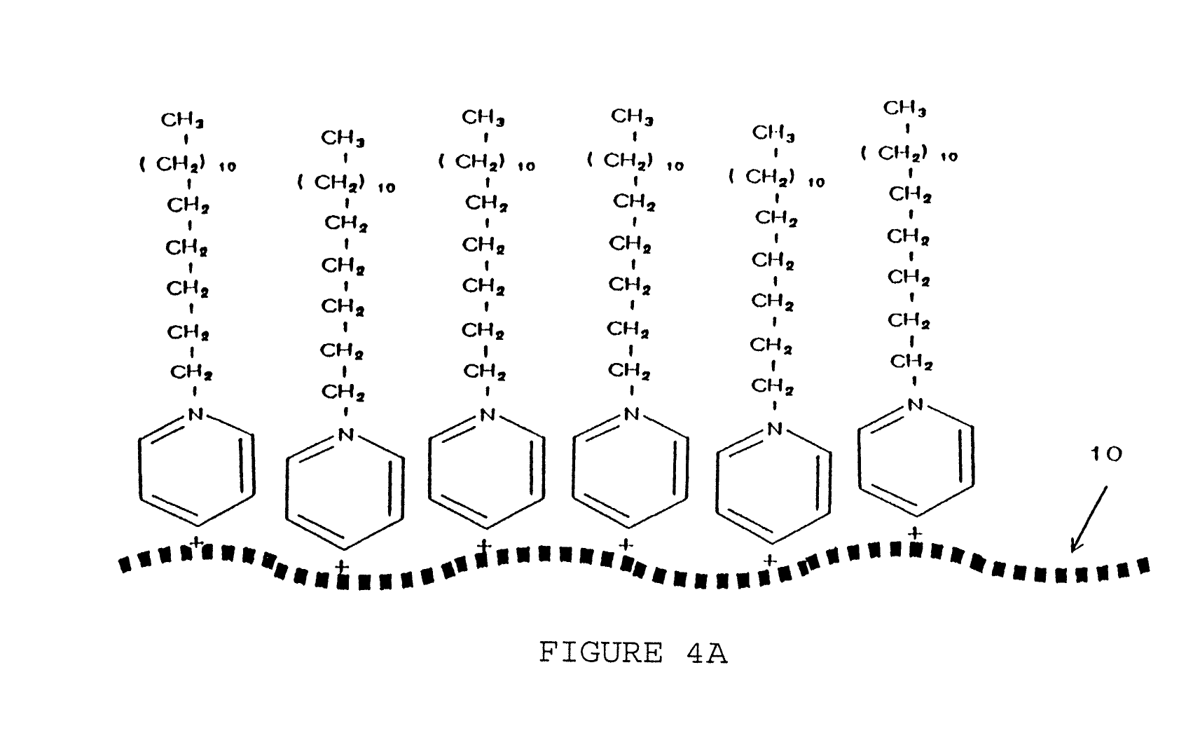

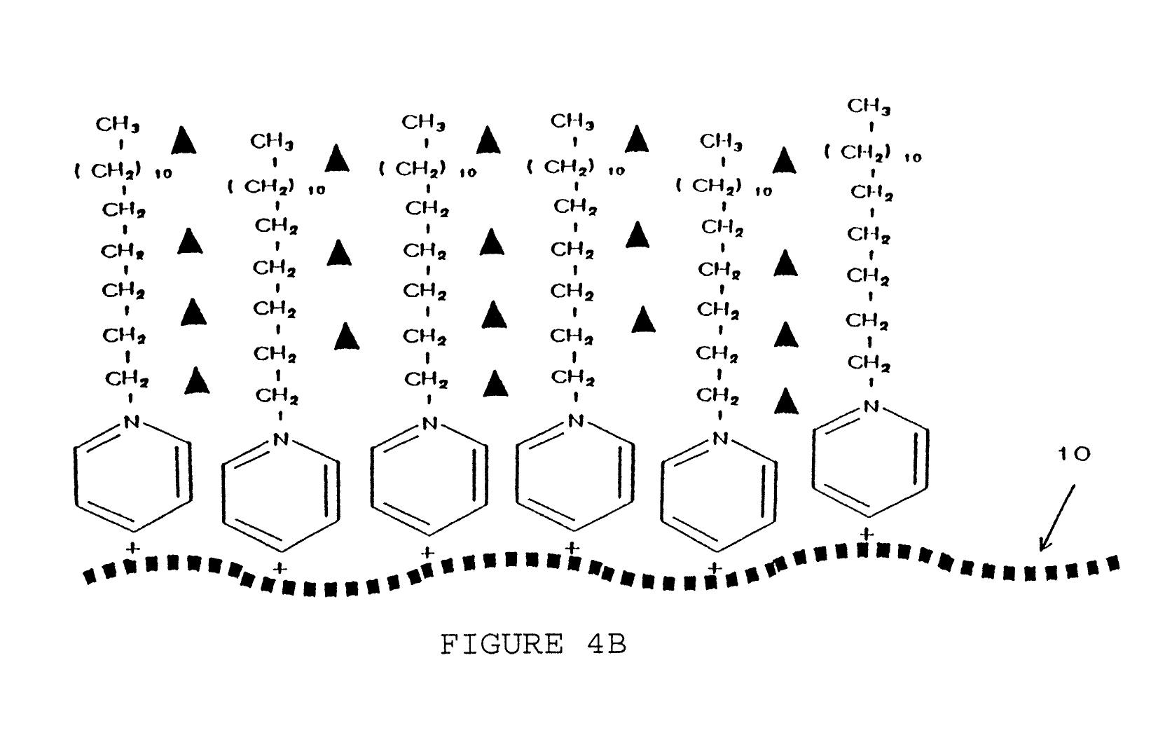

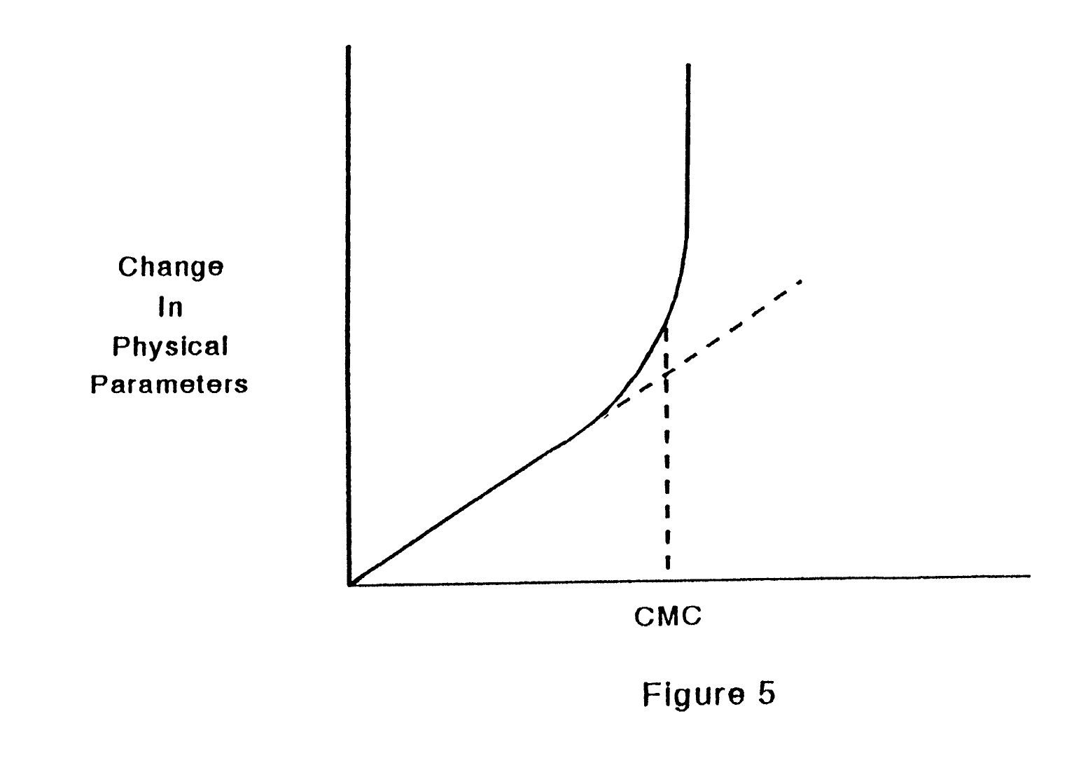

Cetyl pyridinium chloride (CPC) is a quaternary ammonium compound and is a member of a class of cationic surfactants which contain a positively charged quaternary amine and an attached hydrophobic structure. The structure of CPC, which is illustrated in FIG. 1, shows a positively charged nitrogen atom contained within a pyridine ring which in turn is attached to a saturated straight chain alkyl group containing sixteen carbon atoms. Chemically, CPC is classified as an amphiphile. Hence, the hydrophilic head group represented by the pyridine ring is illustrated with the symbol (●), and the hydrophobic tail group is illustrated with the symbol (-). Cetyl pyridinium chloride has a critical micelle concentration (CMC) of 0.9 mM. Thus, at this concentration and above, the molecule is capable of forming molecular aggregations with other CPC molecules. However, this functional characteristic is expressed only under certain conditions. In dilute aqueous solutions of less than 10⁻⁴ or 10⁻⁵ M, the behavior of the CPC amphiphile parallels that of a strong electrolyte and exhibits properties applicable to this invention. Conversely, at higher concentrations, the amphiphile exhibits a marked deviation in molecular and physical behavior. These deviations are observed as changes in structure and manifested as new physical chemical properties. When CPS is prepared at concentrations above 0.9 mM, it is capable of self-association which results in the formation of structured micelles which can function as biological detergents. An example of the CPC micellar structure is shown in FIGS. 2a and b. FIG. 2a is a diagrammatic representation of a spherical micelle cross-section. FIG. 2b depicts the structural formula of CPC and is representative of a cross-sectional plane of the CPC micelle. The hydrophobic chains are buried within the core of the micellar structure and are sequestered and inaccessible to the bulk aqueous phase environment. This structure is clearly differentiated from the structure of a bipolar lipid membrane, such as a liposomal membrane, which is shown only for comparison purposes in FIG. 3. A liposomal membrane has the hydrophobic regions buried within the bipolar membrane structure. These regions are also inaccessible to the bulk aqueous phase environment. This invention utilizes concentrations of amphiphile below the critical micelle concentration to enable the hydrophobic tail regions to function collectively and in concert with other CPC molecules and be oriented toward the bulk aqueous phase. This collective structure is shown in FIG. 4a and provides a hydrophobic surface, positioned distally to the mucin coat. The exposed hydrophobic surface provides a sink in which organic molecules may be sequestered. FIG. 5 is a graph which depicts the plot of the variations in physical properties of an amphiphile as a function of the increasing amphiphile concentration. A pronounced change in physical characteristics signaling the formation of micelles is indicated by the abrupt deviation from the linearity of the response curve as the critical micelle concentration is approached. A change from the amphiphilic state to the micellar state is observed to occur at or near the CMC.

As a result of the increasing concentration, amphiphilic molecules begin to associate. Eventually micelles are formed. Some of the new physical and molecular properties manifested by the micelle are related to the interfacial tension, electrical conductivity, electromotive force, pH, density, specific heat, temperature coefficients or solubility, viscosity, and optical and spectroscopic properties of the solution. These differences in structure and behavioral properties demonstrate that there is a marked contrast between an amphiphile exhibiting properties of an electrolyte (that is existing as freely rotating molecules in solution), and the same amphiphile at higher concentrations above the CMC functioning as a micelle. This invention utilizes amphiphilic molecules in the free state, in the absence of micelles, to facilitate attachment to mucin coated surfaces and provide a continuous hydrophobic coating away from the mucin surface.

This invention focuses on utilizing the positive charge potential of the quaternary nitrogen atom of CPC to accentuate the binding of the amphiphile to mucin coated surfaces. It also utilizes an alkyl chain functionality for the sequestering of organics. The alkyl chain is covalently attached to the pyridinium ring in cetyl pyridinium chloride and functions as a sink for entrapping flavorant molecules, especially ones with volatile properties, such as menthol. Also, therapeutic agents, such as tricaine and ρ-aminobenzoic acid ethyl aster may be sequestered in the sane manner. In order for cetyl pyridinium chloride to function as a trapping agent for the organic molecules, the long alkyl chain must first be made accessible to the intended organic molecules. These chains then must be oriented in a collective linear array such that they can function in concert with neighboring CPC molecules. This concept in shown in FIG. 4a. In this manner, flavorant can become entrapped or intercalated between the alkyl chains and sequestered for an undetermined period of time. The chemistry of the interaction requires that the concentration of cetyl pyridinium chloride be maintained below the critical micelle concentration of 0.9 mM, or 247 ppm. Under these conditions, the structure illustrated in FIG. 4a will predominate and exist independently of any micelle formation. Contrary to the application of this invention, high concentrations of CPC results in micellar formation that would permit strong surfactant like properties to emerge and dominate in solution. The micellar structure would, as dictated by the laws of surface chemistry, present a positively charge pyridinium ion shield as illustrated in FIG. 2b. This structure would not facilitate the sequestering of organic flavorants or therapeutics such as ρ-aminobenzoic acid ethyl ester or tricaine. Thus, a provision of this discloser is that the concentration of CPC be kept below the critical micelle concentration when used by the consumer. This enables the molecules of cetyl pyridinium chloride with the positively charged quaternary ammonium ions and attached alkyl chains to line up in a linear ray at the mucin surface. The alkyl chains provide a contiguous and continuous matrix of hydrophobicity to be exposed outwardly and be positioned distally to the mucin surface. These exposed hydrophobic groups provide for the sequestration of selected organic molecules and make this invention workable.

Once the CMC of cetyl pyridinium chloride is exceeded by a factor of two or more, a very noticeable and marked astringency sensation is observed in the presence of flavorant. In addition, there is a constricting or compressing of the mucus coated tissues in the oral cavity during testing which results in a bitter astringency which is not aesthetically pleasing. Consequently, there are constraints on how high the concentration of cetyl pyridinium chloride may be increased before it lacks utility.

### EXAMPLE 1

The solid breath mint and the therapeutic ρ-aminobenzoic acid ethyl ester formulations utilize CPC at concentrations around the CMC. The concentrations of CPC in the mint is 0.5 mg CPC per 1800 mg total of mint or 0.0277% by weight. If this amount of CPC were to be immediately solubilized in 1.0 ml aqueous media such as saliva, the critical micelle concentration would be quickly achieved and undesirable binding problems would be observed as well as undesirable taste perceptions. However, by incorporating sweeteners like equal, or aspartame, and bulking agents such as sorbitol, the solubilization of CPC is retarded because the mint dissolves slowly. Hence, the critical micelle concentration of CPC is never achieved during actual taste testing.

### BEST MODE

In the manufacture of mints, the CPC is initially solubilized in flavorant. This is an example of solubilizing one organic constituent in another. When these constituents are added to the mint components, the molecules of cetyl pyridinium chloride are freely rotating with the other flavorant organics and are continually mixed around one another. When the mint is slowly solubilized in the oral cavity over a 5 to 10 minute period, the positively charged pyridinium ions and the long hydrophobic tails of CPC in accompaniment with the flavorant molecules bind to the mucin coated surfaces of the oral cavity. Experimentally, it has been observed that the formulation of cetyl pyridinium chloride into peppermint oil followed by sorbitol as a bulking agent plus an added sweetener, such as equal or aspartame, forms a unique mint construct. These additive ingredients work together in a collective manner to provide prolongation of flavor. These effects have been observed and recorded in a blind study by a taste test panel. The results from this study showed that the mint formulation provided a superior, longer tasting, and fresher mint product. The undesirable characteristics of after taste and astringency that may accompany other products are absent from this formulation. The results from the study that utilized the topical anesthetic ρ-aminobenzoic acid ethyl aster were equally convincing.

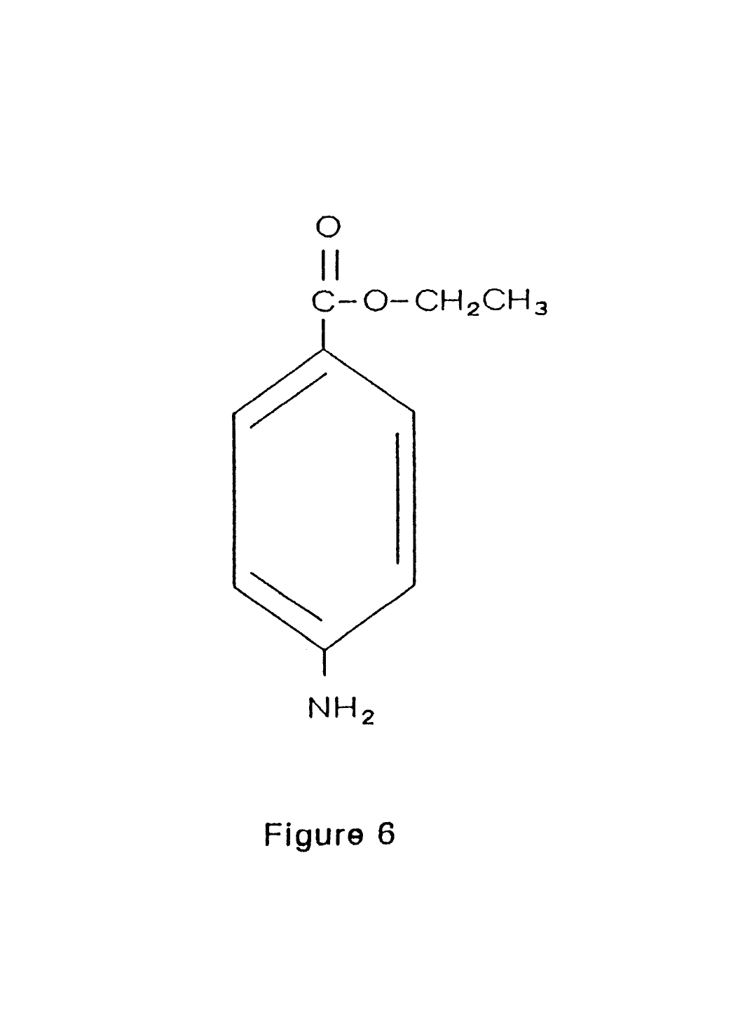

The structural formula of ρ-aminobenzoic acid ethyl aster synonym: ethyl amino benzoate is shown in FIG. 6. Because of its chemical properties,

ρ-aminobenzoic acid ethyl aster can be intercalated between the alkyl chains of cetyl pyridinium chloride. FIG. 4b illustrates how cetyl pyridinium chloride when bound to a mucin surface in an linear array permits ρ-aminobenzoic acid ethyl aster to be interspersed and entrapped between the hydrophobic chains.

### EXAMPLE 2

When a mucin gel is prepared by binding mucin covalently to a cyanogen bromide activated sephrose, a mucin matrix is created that allows binding of cetyl pyridinium chloride.

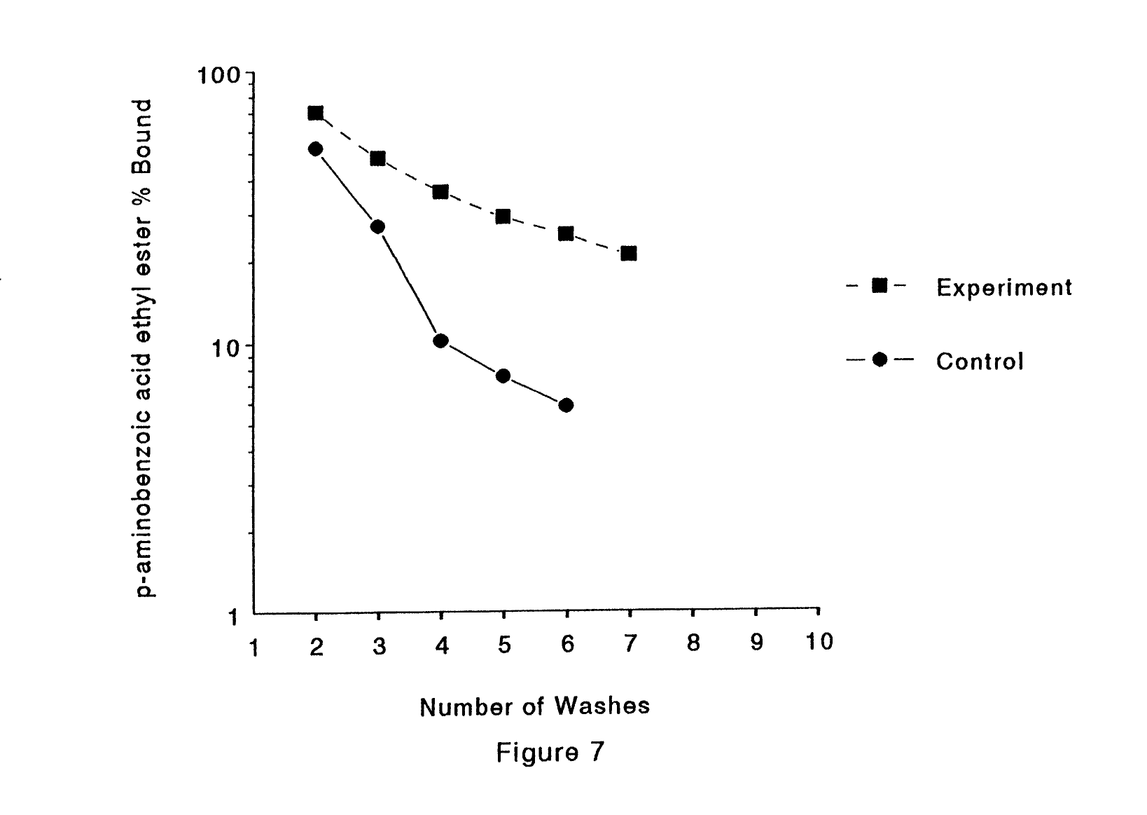

The graph in FIG. 7 illustrates a controlled experiment indicated by the legend: (▴-▴) in which 50 μg of ρ-aminobenzoic acid ethyl ester was added to a mucin gel and then easily washed away. This same figure shows results using an experimental sample as indicated by the legend: (▪-▪) which was prepared o using the same amount (50 μg) of ρ-aminobenzoic acid ethyl ester. The difference between the experimental and control preparation was that in the experimental ρ-aminobenzoic acid ethyl ester was added to the mucin gel along with cetyl pyridinium chloride. The concentration of CPC was below the CMC. This graph illustrates that the control, with only ρ-aminobenzoic acid ethyl ester, washes away from mucin gel in a such more rapid fashion than the combination of ρ-aminobenzoic acid ethyl ester and CPC. The results indicate that cetyl pyridinium chloride has a special adherence property which facilitates binding of the molecule to mucin. Furthermore, a concomitant benefit is realized by retarding the washing away of ρ-aminobenzoic acid ethyl ester. The differences observed in the binding experiment between the control and experimental preparations illustrate an exceptional example of the usefulness of this invention.

The structural formulas of ρ-aminobenzoic acid ethyl ester in FIG. 6 depicts a primary amino group in the para position of the aminobenzoic acid ethyl ester benzene ring. This amino group becomes positively charged as the pH decreases. However, as supported by the data in FIG. 7, even this positive charge is not strong enough to hold the ρ-aminobenzoic acid ethyl ester on the mucin surface for a prolonged period of time. However, cetyl pyridinium chloride has a positive charge that is active and continually present in the pyridinium ring. This positive charge is independent of the pH. This graph illustrates clearly that cetyl pyridinium chloride is functioning as a mucin adhering agent as disclosed in the specification. The fact that ρ-aminobenzoic acid ethyl ester can be held, or bound, or intercalated in CPC is a clear indication that this topical anesthetic can be made to adhere to mucin surfaces utilizing the correct formulation of CPC and active. The binding of ρ-aminobenzoic acid ethyl enter nay thereby be prolonged and the duration of its pharmacological action extended. These effects should have a pronounced benefit for the consumer or the patient who uses ρ-aminobenzoic acid ethyl ester products with the correct and optimum levels of cetyl pyridinium chloride. Another benefit conferred is the time-release leaching of the topical anesthetic agent from the CPC matrix. A further elaboration of the idea disclosed for cetyl pyridinium chloride also maintains for the following quaternary ammonium compounds: cetyl konium chloride, cetyl dimethyl ethyl ammonium bromide, benzyl triethyl ammonium chloride, cetyl trimethyl ammonium para toluene sulfonate, cetyl trimethyl ammonium bromide, distearyl dimethyl ammonium methosulfate, tetra n-butylammonium bromide, myristyl trimethyl ammonium bromide, cetyl dimethyl benzyl ammonium chloride, cetyl pyridinium bromide, cetyl trimethyl ammonium chloride, stearyl dimethyl benzyl ammonium chloride, cetyl trimethyl ammonium stearate, benzal konium chloride, domiphen bromide, and methyl benzethonium chloride.

What is claimed is:

1. A mucin adhering substance, said substance comprising a continuous positively charged hydrophilic head group covalently attached to a hydrophobic tail group:
consisting of a plurality of said substance attachments to mucin coated surfaces;
said substance functioning in concert with other said substances;
whereby, said substances are capable of sequestering organic molecules;
whereby, said organic molecules may leach from said substances;

2. The substance of claim 1 comprising a class of amphiphilic quaternary ammonium compounds.

3. The organic molecules of claim 1 being a therapeutic or comestible.

4. The substance of claim 2 belonging to a class of quaternary ammonium compounds comprising: cetyl konium chloride, cetyl dimethyl ethyl ammonium bromide, benzyl triethyl ammonium chloride, cetyl trimethyl ammonium para toluene sulfonate, cetyl trimethyl ammonium bromide, distearyl dimethyl ammonium methosulfate, tetra n-butylammonium bromide, myristyl trimethyl ammonium bromide, cetyl dimethyl benzyl ammonium chloride, cetyl pyridinium bromide, cetyl trimethyl ammonium chloride, stearyl dimethyl benzyl ammonium chloride, cetyl trimethyl ammonium stearate, benzal konium chloride, domiphen bromide, methyl benzethonium chloride.

5. The comestible of claim 3 being a menthol flavorant

6. The therapeutic of claim 3 being an anesthetic, analgesic, antibiotic, antifungal, antitoxin, antihistamine, antineoplastic, antihelminthic, bronchial dilator, or ophthalmic.
